(19) United States
(12) Patent Application Publication
(10) Pub. No.: US 2010/0280331 A1
(43) Pub. Date: Nov. 4, 2010

(54) Method and apparatus for non-invasively estimating body core temperature

(75) Inventors: Jonathan Kaufman, Leonardtown, MD (US); Stephen M. Coleman, Lusby, MD (US)

Correspondence Address: Department of the Navy;(Naval Air Warfare Center -Aircraft Division), 47076 Lijencreantz Road, B 435, PATUXENT RIVER, MD, 20670, US

(73) Assignee: Department of the Navy

(21) Appl. No.: 11/998,863

(22) Filed: Nov. 28, 2007

(51) Int. Cl. A61B 5/00

(52) U.S. Cl. 600/301

(57) ABSTRACT

Non-invasive methods and devices are disclosed to derive estimates of body Core Temperature from external sensors that provide electrocardiograph (ECG) data, and Mean Skin Temperature data. The ECG and Mean Skin Temperature are input to a model that provides estimates of Core Temperature temperature. The model is derived regressively from ECG, Mean Skin Temperature and Core Temperature data obtained from a number of test subjects. A monitoring device may be used, for example, to trigger an alarm, display Core Temperature data to the device wearer or to a remote monitoring station, or to activate an emergency temperature control system or device.

### GOVERNMENT INTEREST

The invention described herein may be manufactured, licensed, and used by or for the U.S. Government.

### TECHNICAL FIELD

The present invention relates in general to predicting thermal strain in humans and other organisms, and more particularly to a method and apparatus for non-invasively estimating body Core Temperature.

### BACKGROUND

“Core temperature” is the operating temperature of a body that exists in deep structures such as the liver, heart, and brain, in comparison to temperatures of peripheral tissues. Temperature control (i.e., thermoregulation) is part of a homeostatic mechanism that maintains the body's set point at its optimum operating temperature. In humans, the nominal optimum Core Temperature is typically said to be 37.6° C. (99.6° F.). Thermal stress is a condition that occurs when the body deviates enough from its optimum Core Temperature to disrupt vital functions. Deviations of only a few degrees from the nominal optimum Core Temperature can be dangerous and possibly fatal if a severe thermal strain condition persists for too long. All too often, a person suffering from thermal stress does not know how much danger they are in until it is too late.

Skin temperature and oral temperature readings do not correlate well with body Core Temperature. Body Core Temperature measurements are thus obtained through internal measurements taken at the rectum, esophagus, pulmonary artery, urinary bladder, or tympanic membrane. Of these, rectal temperature measurement is the most frequently relied upon method. Some drawbacks with rectal temperature measurement are that it is invasive and uncomfortable and can result in rectal perforation if not performed correctly.

Various methods have been tried to approximate Core Temperature from non-invasive temperature measurements, including measurements taken at the armpit (axilary) or forehead/temporal artery, infrared (non-contact) measurement of the tympanic membrane, and supralingual temperature measurement. Unfortunately, these alternative methods lack sufficient accuracy for most applications. Recently, radio telemetry “thermometer pills” have been investigated. While thermometer pills provide an accurate reading of internal body temperatures as they travel through the digestive tract, their readings can be influenced by the temperature of ingested food and drink. This is a serious drawback since cold or hot drinks are a first line of defense against thermal stress. In addition, thermometer pills have been reported to cause digestive problems in some individuals. Thus, rectal temperature measurement remains the “gold standard” for determining body Core Temperature and there is a need for a non-invasive and accurate method and apparatus for measuring body Core Temperature. Embodiments of the present invention address these concerns.

### SUMMARY

In general, in one aspect, a method of estimating body Core Temperature includes determining Heart Rate Variability, determining Mean Skin Temperature and calculating body Core Temperature based on data including the determined Heart Rate Variability and Mean Skin Temperature. In general, in another aspect, the method includes employing an empirically derived model to calculate the estimates of body Core Temperature. In general, in another aspect, the model to calculate the estimates of body Core Temperature includes one or more parameters derived by a regression analysis performed on data collected from test subjects. In yet another aspect, the method of estimating body Core Temperature includes performing a spectral analysis on the Heart Rate Variability data

In general, in another aspect, an apparatus for estimating the Core Temperature of a body includes a non-invasive sensor to detect cardiac data from the body, a non-invasive sensor to detect temperature data from the body, at least one processor programmed to execute instructions to derive Heart Rate Variability data from the Heart Rate data and to derive Mean Skin Temperature data from the temperature data detected from the body, and further to derive an estimate of body Core Temperature from data comprising the Heart Rate Variability and Mean Skin Temperature data.

### BRIEF DESCRIPTION OF THE DRAWINGS

FIG. 1 shows an example of a QRS wave obtained by a conventional ECG.

FIG. 2 shows an example of a cardiac tachogram.

FIG. 3 shows an example of a power spectral density distribution of the tachogram of FIG. 2.

FIG. 4 shows a diagram of the placement of biologic sensors and a sensor embedded vest, according to an embodiment of the present invention.

FIG. 5 shows a simplified flow diagram of the operation of an embodiment according to the present invention.

FIG. 6 shows a simplified block diagram of a wireless remote Core Temperature monitor according to an embodiment of the present invention.

### DETAILED DESCRIPTION

In the following detailed description, reference is made to the accompanying drawings which form a part hereof, and in which are shown by way of illustration specific embodiments in which the invention, as claimed, may be practiced. The invention may, however, be embodied in many different forms and should not be construed as limited to the embodiments set forth; rather, these embodiments are provided so that this disclosure will be thorough and complete, and will fully convey the scope of the invention to those skilled in the art. As will be appreciated by those of skill in the art, the present invention may be embodied in methods and apparatuses.

### Detection Apparatus

In general, embodiments according to the present invention derive body Core Temperature estimates from external sensors that provide electrocardiograph (ECG) data, and Mean Skin Temperature data. ECG data may be obtained by instrumentation ranging from a simple one or two lead “sports” heart rate monitor, such as those worn by endurance athletes, to a standard 12 lead hospital ECG. Mean Skin Temperature data, by convention, involves measuring skin temperatures from several locations on the body, such as the chest, upper arm, thigh and calf/shin. In alternative embodiments, a greater or lesser number of skin temperature sensors may be employed.

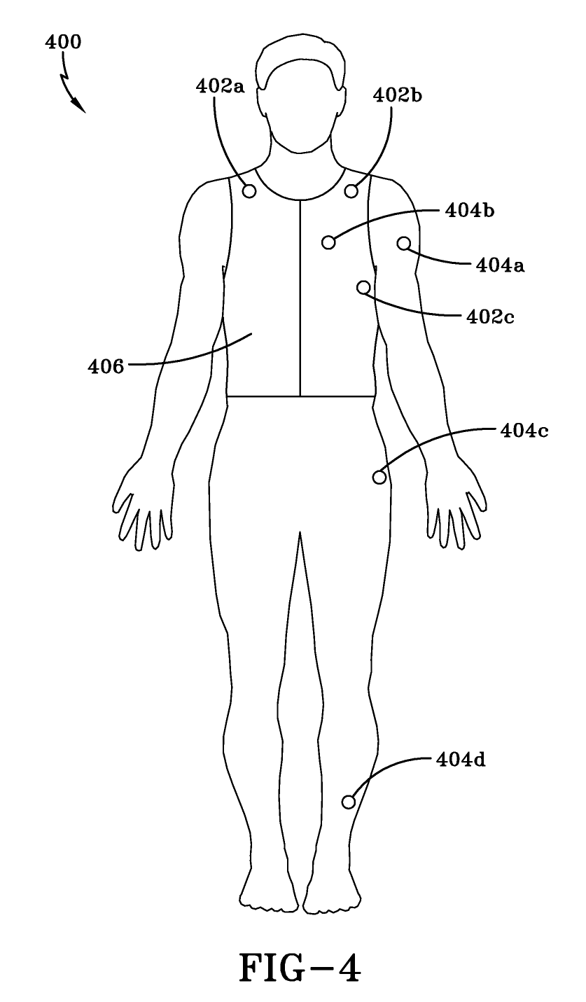

FIG. 4 illustrates the placement of biological sensors in an embodiment of a non-invasive body Core Temperature apparatus 400 according to the present invention. In this example, two cardiac leads 402a and 402b, which include appropriate cardiac electrodes, and a ground lead 402c, are provided. Cardiac leads 402a and 402b and ground lead 402c are incorporated into a vest 406 that may be worn by a person whose Core Temperature is to be monitored. Vest 406 should be worn next to the skin to provide good skin-to-sensor contact.

The placement of cardiac leads 402 in FIG. 4 on opposite sides of the upper chest is merely illustrative and leads 402 may be positioned in a number of different ways to capture r-wave data, as would be known by those of skill in the art. Likewise, a variety of other sensors may be employed to detect r-wave data in alternative embodiments. For example, r-wave data may be detected by infrared sensors, audio frequency sensors, pneumatic sensors, accelerometers, or similar.

Several skin temperature sensors 404 are generally needed to determine the Mean Skin Temperature of the body. Four sensors 404 are shown in this embodiment: a skin temperature sensor 404a positioned on the left upper arm, a skin temperature sensor 404b positioned on the left side of the chest, a skin temperature sensor 404c positioned on the left thigh and a skin temperature sensor 404d positioned on the left calf of the person being monitored. Temperature sensors 404 are conventionally positioned on the left side of the body, however, right side temperature sensor placement may be used, if desired. In this embodiment IR thermocouple temperature sensors have been employed. IR thermocouples have the advantage of being self-powered, relying only on the incoming infrared radiation to produce an output signal (in the my range) through thermoelectric effects. The output from IR thermocouple sensors 404 is sufficiently linear to closely match the my vs. temperature curve of a given thermocouple type, such as the “J-type” thermocouple commonly used for body temperature measurements. In alternative embodiments, temperature sensors such as thermistors and resistance temperature devices (RTD's) may be employed. In still other alternative embodiments, non-contact sensors may be used, including but not limited to infrared, thermal imaging, and the like, although such sensors are generally less accurate. Typically, mean skin temperature (first suggested by Ramanathan, 1964) is given by MST=0.3*(chest temp+upper arm temp)+0.2*(thigh temp+calf/shin temp), though other equations using 3-14 separate skin sites (e.g., abdomen, scapula, hand, lower back) are also employed.

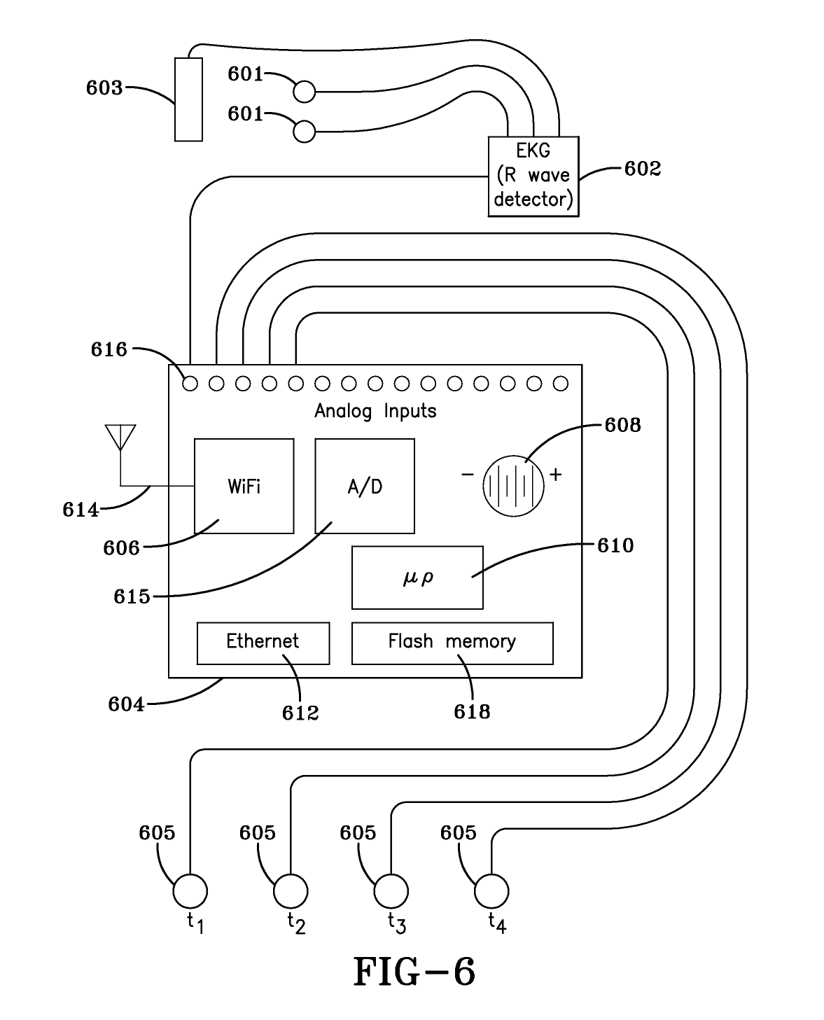

Data from sensors 402 and 404 may be processed by a monitoring device carried by the monitored subject or transmitted to a remote monitoring station for processing. For example, a wireless Core Temperature monitor 600 is shown in FIG. 6, described below. Core Temperature estimates may be displayed, recorded or used to trigger alarms. In some embodiments, a Core Temperature that falls outside of a predetermined normal range may be used to activate thermal control devices such as fans, air conditioners, cooling jackets, heating elements and the like, to cool or heat the body, as appropriate.

While a vest 406 provides a convenient carrier for sensors and monitoring apparatus modules, other types of garments may likewise be used to carry sensors and monitoring modules in embodiments according to the present invention, including undergarments, dive suits, flight suits, g-suits, corsets, and the like.

### Calculating Heart Rate Variability

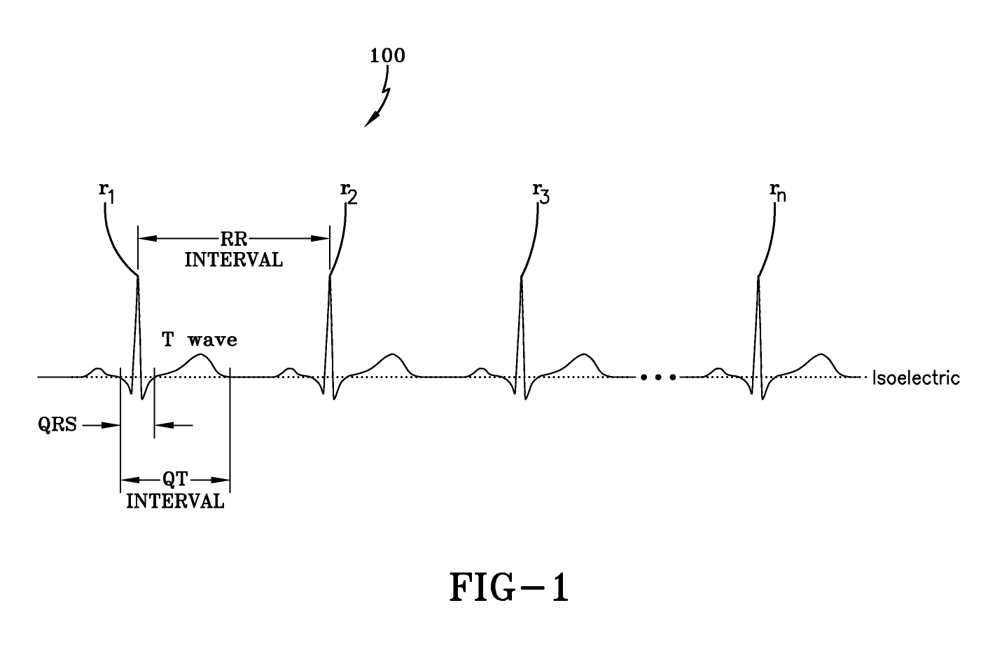

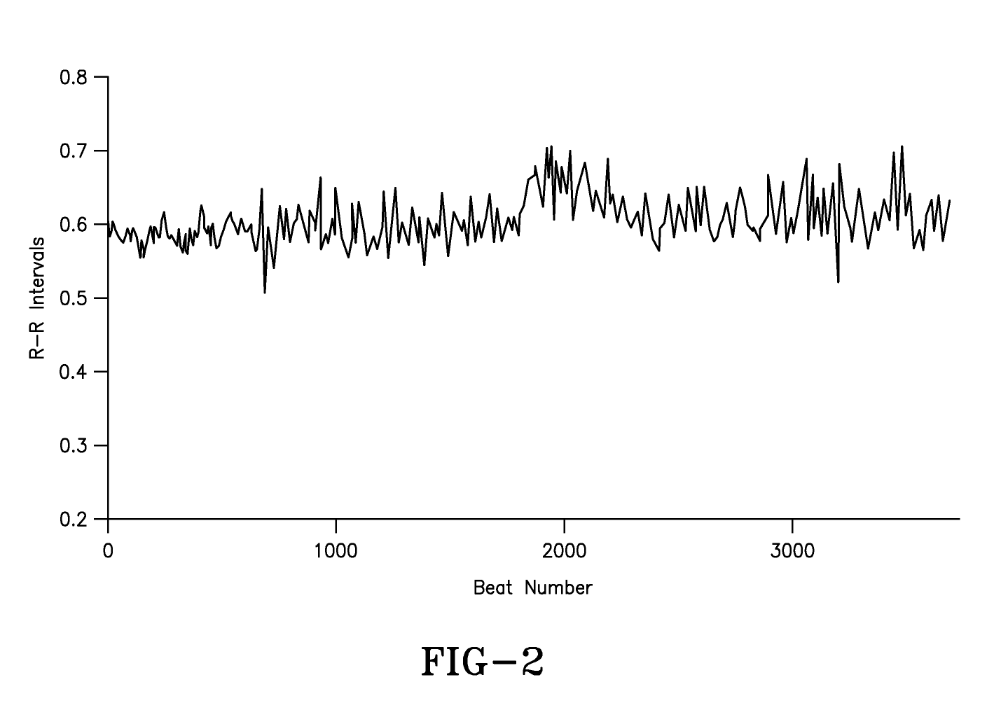

FIG. 1 shows a typical QRS wave obtained by a conventional ECG. Heart rate variability (Heart Rate Variability) is defined as the time difference or variation between successive r-wave intervals. To determine Heart Rate Variability, the cardiac data from ECG leads 402 is processed to obtain a time series of r waves, r₁, r₂, . . . , rₙ at times tₘ. r-r intervals are determined by subtracting (tᵣₙ₊₁−tᵣₙ) for each rₙ. FIG. 2 shows a time varying tachogram in which the y-axis plots r wave intervals and the x-axis the total number of beats.

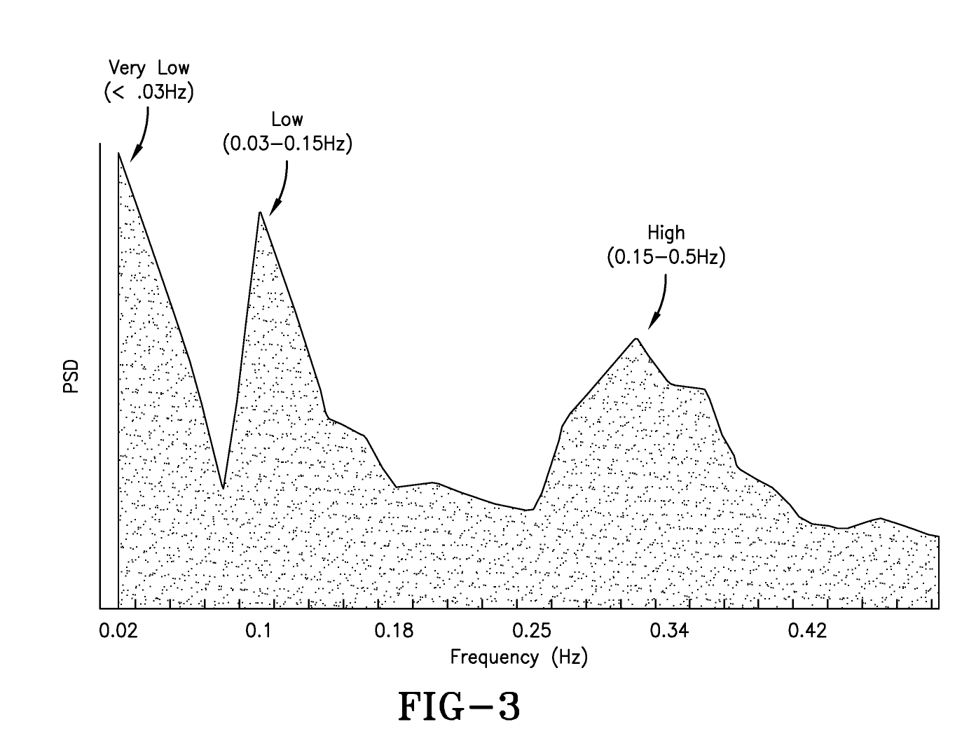

Spectral analysis of the tachogram data of FIG. 2 transforms the signal from the time domain to the frequency domain. FIG. 3 shows a power spectral density distribution of the tachogram data of FIG. 2. In FIG. 3, the y axis amplitudes represent the power of spectral components and are presented in absolute units (milliseconds squared). The Heart Rate Variability spectrum generally contains three major spectral components: High Frequency oscillations (about 0.15-0.4 Hz), which are mainly associated with mechanical and reflex components of respiratory activity, Low Frequency oscillations (about 0.04 to 0.15 Hz), influenced primarily by the vagus and cardiac sympathetic nerves (i.e., vasomotor activity), including feedback loops associated with the baroreflex; and Very Low Frequency oscillations (less than or equal to about 0.04 Hz) which depend on several factors including slow respiratory-based oscillations, effects of blood volume changes and thermoregulatory fluctuations in vasomotor status. A ULF component is also sometimes identified for oscillations less than or equal to about 0.003 Hz but requires very long term ECG recordings for reliable observation, typically a period of 24 hours. It should be noted that these major spectral components are approximate ranges and that the frequency specified do not represent exact cut offs. For example, in some embodiments the VLF frequency range may be less than or equal to about 0.03 Hz.

Heart Rate Variability may be calculated by a number of different techniques. Regardless of the technique employed, care should be taken to ensure that spectral data is not distorted or lost by using an inappropriate transform, improper windowing or filtering. In particular, tachogram data is a biologic signal that is essentially nonstationary. A spectral analysis technique that operates well on nonstationary signals should therefore be selected.

### Estimating Core Temperature

Although, as noted, Mean Skin Temperature is not a reliable indicator by itself of body Core Temperature, and there is no direct relationship between Heart Rate Variability and body Core Temperature, when Heart Rate Variability and Mean Skin Temperature data sets are processed according to embodiments of the present invention, reliable estimates of Core Temperature may be obtained.

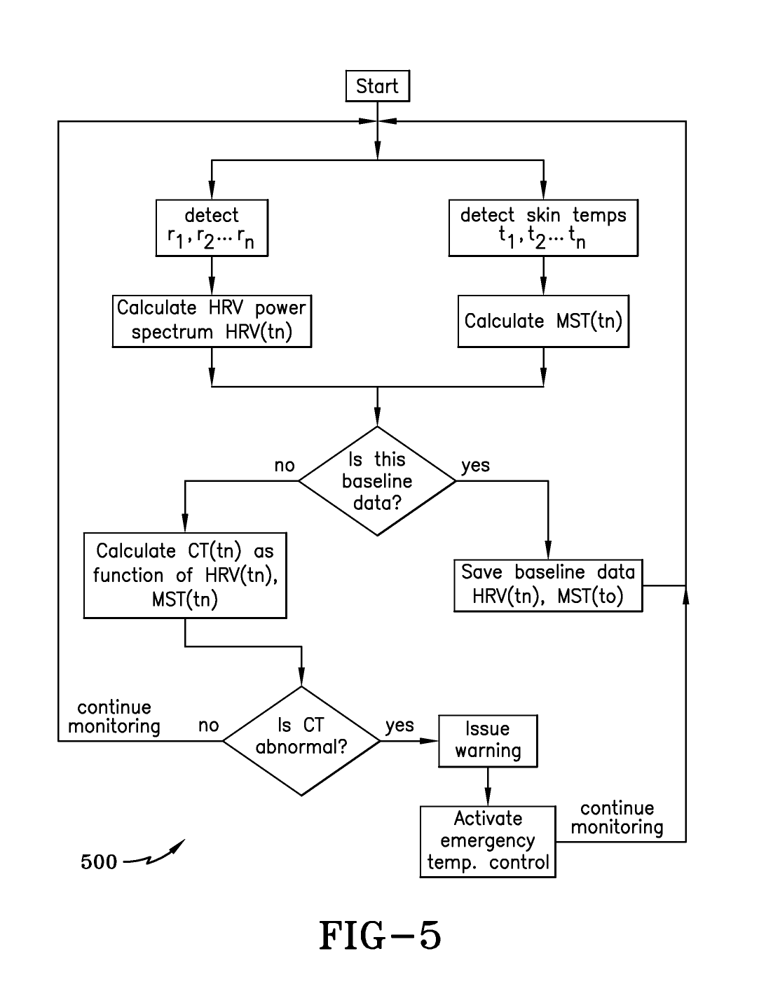

FIG. 5 shows an exemplary flow diagram of the operation of a Core Temperature monitor according to an embodiment of the present invention. At 502, baseline and initialization data is recorded. The data includes the measurement subject's height and weight and the ambient temperature, i.e., the temperature to which the skin is exposed. Including the subject's height and weight provides better estimates of Core Temperature. Ambient temperature may be sensed by any suitable temperature sensor, such as an IR thermocouple, thermocouple, thermistor, RTD, or the like. After the initial values have been entered, the system begins to gather r-wave data and skin temperature data at 504. R-wave data is recorded until there is a tachogram of sufficient length to perform a spectral analysis that yields at least the VLF portion of the Heart Rate Variability spectrum. In general, for spectral computations, the lowest frequency of interest determines the record length. For example, if a sample is evaluated for VLF content, tachogram data of at least 33 seconds in duration is needed. It was determined experimentally that a 5 minute tachogram will provide enough data to reliably extract the VLF range oscillations, in most circumstances.

If it is determined at 508 that this is an initial run, baseline values for Mean Skin Temperature, HR, Heart Rate Variability and ambient temperature are recorded at 510 to be used as a reference point.

At 508, an estimate of Core Temperature is then calculated based on the following model:

CT=a0+a1*HR+a2*MST+a3*ambient temp+a4*height*weight+a5*HR*MST+a6*HR*ambient temp+a7*HR*height*weight+a8*VLF*MST+a9*VLF*ambient temp+a10*VLF*height*weight+a11*MST*ambient temp+a12*HR*VLF
where CT is Core Temperature, HR is Heart Rate, MST is Mean Skin Temperature, VLF is a very low frequency spectral component of Heart Rate Variability and a0, a1, a2 . . . a12 are model parameters.

The model parameters a0, a1, a2 . . . a12 are derived empirically by a regression analysis. In general, a regression analysis examines the relation of a dependent variable to specified independent variables. In this case, the independent variables of Heart Rate, height, weight, ambient temperature, Heart Rate Variability and Mean Skin Temperature were evaluated to determine their relation to actual Core Temperature readings (rectal temperature). To establish the model parameters, Heart Rate, height, weight, Heart Rate Variability, Mean Skin Temperature and Core Temperature values were observed and recorded for more than 60 test subjects. Test subjects were observed in a variety of thermal environments in order to obtain a wide range of Core Temperature values, including Core Temperature values in the hyperthermia and hypothermia ranges. The regression analysis of this data yielded values for a0 . . . a12 as follows: a0=0.084198; a1=0.230214; a2=−0.25538; a3=−0.09152; a4=0.045715; a5=−0.09063; a6=−0.06626; a7=−0.16768; a8=0.052633; a9=0.052633; a10=−0.08471; a11=0.211613; and a12=0.056554.

Test subjects were drawn from a pool of Navy and Marine personnel and professional and volunteer civilian firefighters. The model developed in this embodiment according to the present invention matches the test data very closely. As more test data is recorded over a broader spectrum of the population the model may be expected to change.

After a Core Temperature estimate has been derived, the system determines whether the Core Temperature is within the range of normal. If the Core Temperature is in normal range, the system continues monitoring. In some embodiments, Core Temperature values may be displayed remotely and/or on a display available to the wearer of the monitoring device. If an abnormal Core Temperature has been detected, the system may issue a warning to the operator, the wearer of the monitoring device, or both. Additionally, in some embodiments, the system may be programmed to activate an emergency temperature control device when the Core Temperature falls outside of a predetermined range. For example, a peltier cooling element, a water cooling jacket, a blower fan, or the like, may be incorporated in a suit worn by a person whose Core Temperature has become elevated, such as a firefighter. Alternatively, a chemical or electrical heating apparatus may be incorporated in a suit worn by a person whose Core Temperature has fallen, such as a diver.

### Hardware Prototype

FIG. 6 shows an embodiment according to the present invention of a wireless remote Core Temperature monitor 600, and which optionally provides the capability of monitoring other biologics such as electromyographic data. Wireless remote Core Temperature monitor 600 includes an ECG module 602 to which are connected two ECG electrodes 601 and a ground strap 603 and a transceiver controller module 604 that receives and processes raw analog data, processes the data and transmits Core Temperature and other biologic data wirelessly to a remote station. Wireless remote Core Temperature monitor 600 is suitable for any application where remote Core Temperature monitoring is desired. Electrodes 601 may be attached to the body in any locations where cardiac r-wave potential differences may be detected, such as the left and right sides of the chest. Ground strap 603 is attached to the body in a third location, such as along the waistline, to obtain a reference potential. The leads from ECG sensors 601 to ECG module 602 are shielded to minimize pickup of noise.

ECG module 602 provides opto-isolation, amplification and signal processing of the raw ECG data from ECG sensors 601. ECG module 602 outputs a continuous stream of amplified, processed r-wave analog signals r₁, r₂, r₃, . . . , rₙ via a shielded cable that is received at an analog input channel 616 of a transceiver controller module 604. While ECG module 602 is shown as a separate module (and is powered by an onboard battery which is not illustrated) in alternative embodiments, ECG module 602 and transceiver controller module 604 may be integrated into a single module.

Wireless remote Core Temperature monitor 600 also includes skin temperature sensors 605 which, in this embodiment, are self-powered IR thermocouples. Four temperature sensors 605 are provided for attachment to the arm, chest, calf/shin and thigh, for conventional Mean Skin Temperature measurements. The leads for temperature sensors 605 are shielded to minimize pickup of noise. An additional temperature sensor 607 is also provided for measuring room temperature or temperature inside thermal protective gear such as a HAZMET suit or a firefighter's suit.

Transceiver controller module 604 is powered by an onboard battery 608 and provides multiple analog input channels 616, an analog-to-digital converter 615, an Ethernet port 612, a wireless transceiver module 606, a microprocessor 610 and flash memory 618. Analog input channels 616 are digitized by an onboard analog-to-digital converter 615. While transceiver module 606 is a standard “wifi” 802.11 compliant communication link that employs conventional TCP/IP protocols, in alternative embodiments other types of wired or wireless communication links and/or protocols may be employed, depending on the needs of a particular application, including a wide variety of analog or digital radio frequency devices, or devices employing infrared, inductive coupling, ultrasonic, or similar.

### CONCLUSION

As has been shown, embodiments of the present invention provide a method and apparatus for non-invasively estimating body Core Temperature. Embodiments according to the present invention provide a reliable and non-invasive method and apparatus for measuring body Core Temperature and will find use in a wide variety of applications where exposure to prolonged heat and/or cold may lead to thermal stress, including use in connection with firefighting as well as in applications where protective gear such as HAZMET or chemical-biological protective suits may cause overheating after extended use. Similarly, embodiments according to the present invention may be used to monitor Core Temperature of individuals engaged in vigorous activity where overheating may sometimes occur, such as football, marathon running, and the like, and may be incorporated into apparel designed for such activities. Embodiments according to the present invention will also find use in other environments where Core Temperature monitoring is desired, such as in hospitals, emergency rooms, ambulances and medivac units. Other embodiments may also be incorporated in survival kits and related severe weather outdoor survival gear including marine survival suits, mountain climbing gear, dive suits, aircraft pressure suits, space suits, and the like.

A number of embodiments of the invention defined by the following claims have been described. Nevertheless, it will be understood that various modifications to the described embodiments may be made without departing from the spirit and scope of the claimed invention. Accordingly, other embodiments are within the scope of the invention, which is limited only by the following claims.

What is claimed is:

1. A method of estimating body Core Temperature, comprising:
determining Heart Rate Variability;
determining Mean Skin Temperature; and
calculating body Core Temperature based on data comprising the determined Heart Rate Variability and Mean Skin Temperature.

2. The method of estimating body Core Temperature according to claim 1, wherein estimating body Core Temperature based on the Heart Rate Variability and Mean Skin Temperature comprises employing an empirically derived model.

3. The method of estimating body Core Temperature according to claim 2, wherein the model comprises one or more parameters and the one or more parameters are derived by a regression analysis performed on data collected from test subjects.

4. The method of estimating body Core Temperature according to claim 3 wherein the data collected from test subjects comprises Heart Rate Variability, Mean Skin Temperature and Core Temperature.

5. The method of estimating body Core Temperature according to claim 1, wherein a spectral analysis is performed on the Heart Rate Variability data.

6. The method of estimating body Core Temperature according to claim 5, wherein the spectral analysis comprises extracting a frequency range from the Heart Rate Variability data spectrum.

7. The method of estimating body Core Temperature according to claim 6, wherein the frequency range extracted from the Heart Rate Variability data spectrum comprises a Very Low Frequency range.

8. The method of estimating body Core Temperature according to claim 1 wherein the data for calculating body Core Temperature comprises a Heart Rate.

9. The method of estimating body Core Temperature according to claim 1 wherein the data for calculating body Core Temperature comprises a body size.

10. The method of estimating body Core Temperature according to claim 3 wherein the body size comprises a body height and body weight.

11. The method of estimating body Core Temperature according to claim 5, wherein the model comprises substantially the following calculation:
CT=a0+a1*HR+a2*MST+a3*ambient temp+a4*height*weight+a5*HR*MST+a6*HR*ambient temp+a7*HR*height*weight+a8*VLF*MST+a9*VLF*ambient temp+a10*VLF*height*weight+a11*MST*ambient temp+a12*HR*VLF,
where CT is Core Temperature, HR is Heart Rate, MST is Mean Skin Temperature, VLF is a very low frequency spectral component of Heart Rate Variability and a0, a1, a2 a12 are model parameters.

12. An apparatus for estimating the Core Temperature of a body, comprising:
a non-invasive sensor to detect cardiac data from the body;
a non-invasive sensor to detect temperature data from the body,
at least one processor programmed to execute instructions to:
derive Heart Rate Variability data from the cardiac data and to derive Mean Skin Temperature data from the temperature data detected from the body, and further to derive an estimate of body Core Temperature from data comprising the Heart Rate Variability and Mean Skin Temperature data.

13. The apparatus according to claim 12, wherein the sensor to detect cardiac data comprises an electrocardiograph sensor.

14. The apparatus according to claim 12, wherein the cardiac data comprise r-waves.

15. The apparatus according to claim 12, wherein a power spectrum is calculated from the Heart Rate Variability data.

16. The apparatus according to claim 12 wherein the estimate of Core Temperature data from data comprising the Heart Rate Variability and Mean Skin Temperature data is obtained from a model.

17. The apparatus according to claim 16 wherein the model is derived from a regression analysis of empirical data comprising Heart Rate Variability, Mean Skin Temperature and Core Temperature measurements.

18. The apparatus according to, claim 12 further comprising a wireless transceiver to transmit data to a remote monitoring station.

19. The apparatus according to claim 12 wherein the model comprises substantially the following calculation:
CT=a0+a1*HR+a2*MST+a3*ambient temp+a4*height*weight+a5*HR*MST+a6*HR*ambient temp+a7*HR*height*weight+a8*VLF*MST+a9*VLF*ambient temp+a10*VLF*height*weight+a11*MST*ambient temp+a12*HR*VLF,
where CT is Core Temperature, HR is Heart Rate, MST is Mean Skin Temperature, VLF is a very low frequency spectral component of Heart Rate Variability and a0, a1, a2 a12 are model parameters.

20. The apparatus according to claim 12 wherein the temperature data from the body comprises skin temperature.

21. The apparatus according to claim 20 wherein the skin temperature is detected by a plurality of external temperature sensors.

22. The apparatus according to claim 12 wherein the plurality of external sensors comprise an IR thermocouple.
